SEQUENCE LISTING (6 sequences)

SEQ ID NO: 1 - protein, 551 aa, Homo sapiens
  MISC_FEATURE (1)..(551): human NF- B p65 subunit isoform 1
MDELFPLIFPAEPAQASGPYVEIIEQPKQRGMRFRYKCEGRSAGSIPGERSTDTTKTHPTIKINGYTGPGTVRISLVTKDPPHRPHPHELVGKDCRDGFYEAELCPDRCIHSFQNLGIQCVKKRDLEQAISQRIQTNNNPFQVPIEEQRGDYDLNAVRLCFQVTVRDPSGRPLRLPPVLSHPIFDNRAPNTAELKICRVNRNSGSCLGGDEIFLLCDKVQKEDIEVYFTGPGWEARGSFSQADVHRQVAIVFRTPPYADPSLQAPVRVSMQLRRPSDRELSEPMEFQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 2 - protein, 548 aa, Homo sapiens
  MISC_FEATURE (1)..(548): human NF-KB p65 subunit isoform 2
MDELFPLIFPAEPAQASGPYVEIIEQPKQRGMRFRYKCEGRSAGSIPGERSTDTTKTHPTIKINGYTGPGTVRISLVTKDPPHRPHPHELVGKDCRDGFYEAELCPDRCIHSFQNLGIQCVKKRDLEQAISQRIQTNNNPFQEEQRGDYDLNAVRLCFQVTVRDPSGRPLRLPPVLSHPIFDNRAPNTAELKICRVNRNSGSCLGGDEIFLLCDKVQKEDIEVYFTGPGWEARGSFSQADVHRQVAIVFRTPPYADPSLQAPVRVSMQLRRPSDRELSEPMEFQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 3 - protein, 482 aa, Homo sapiens
  MISC_FEATURE (1)..(482): human NF-KB p65 subunit isoform 3
MDELFPLIFPAEPAQASGPYVEIIEQPKQRGMRFRYKCEGRSAGSIPGERSTDTTKTHPTIKINGYTGPGTVRISLVTKDPPHRPHPHELVGKDCRDGFYEAELCPDRCIHSFQNLGIQCVKKRDLEQAISQRIQTNNNPFQVPIEEQRGDYDLNAVRLCFQVTVRDPSGRPLRLPPVLSHPIFDNRAPNTAELKICRVNRNSGSCLGGDEIFLLCDKVQKEDIEVYFTGPGWEARGSFSQADVHRQVAIVFRTPPYADPSLQAPVRVSMQLRRPSDRELSEPMEFQYLPDTDDRHRIEEKRKRTYETFKSIMKKSPFSGPTDPRPPPRRIAVPSRSSASVPKPAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 4 - protein, 448 aa, Homo sapiens
  MISC_FEATURE (1)..(448): human NF-KB p65 subunit isoform 4
MDELFPLIFPAEPAQASGPYVEIIEQPKQRGMRFRYKCEGRSAGSIPGERSTDTTKTHPTIKINGYTGPGTVRISLVTKDPPHRPHPHELVGKDCRDGFYEAELCPDRCIHSFQNLGIQCVKKRDLEQAISQRIQTNNNPFQVPIEEQRGDYDLNAVRLCFQVTVRDPSGRPLRLPPVLSHPIFDNRAPNTAELKICRVNRNSGSCLGGDEIFLLCDKVQKEDIEVYFTGPGWEARGSFSQADVHRQVAIVFRTPPYADPSLQAPVRVSMQLRRPSDRELSEPMEFQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQRPPDPAPAPLGAPGLPNGLLSGDEDFSSIADMDFSALLSQISS

SEQ ID NO: 5 - protein, 549 aa, Mus musculus
  MISC_FEATURE (1)..(549): mouse NF-KB p65 subunit
MDDLFPLIFPSEPAQASGPYVEIIEQPKQRGMRFRYKCEGRSAGSIPGERSTDTTKTHPTIKINGYTGPGTVRISLVTKDPPHRPHPHELVGKDCRDGYYEADLCPDRSIHSFQNLGIQCVKKRDLEQAISQRIQTNNNPFHVPIEEQRGDYDLNAVRLCFQVTVRDPAGRPLLLTPVLSHPIFDNRAPNTAELKICRVNRNSGSCLGGDEIFLLCDKVQKEDIEVYFTGPGWEARGSFSQADVHRQVAIVFRTPPYADPSLQAPVRVSMQLRRPSDRELSEPMEFQYLPDTDDRHRIEEKRKRTYETFKSIMKKSPFNGPTEPRPPTRRIAVPTRNSTSVPKPAPQPYTFPASLSTINFDEFSPMLLPSGQISNQALALAPSSAPVLAQTMVPSSAMVPLAQPPAPAPVLTPGPPQSLSAPVPKSTQAGEGTLSEALLHLQFDADEDLGALLGNSTDPGVFTDLASVDNSEFQQLLNQGVSMSHSTAEPMLMEYPEAITRLVTGSQRPPDPAPTPLGTSGLPNGLSGDEDFSSIADMDFSALLSQISS

SEQ ID NO: 6 - protein, 550 aa, Rattus norvegicus
  MISC_FEATURE (1)..(550): rat NF-KB p65 subunit
MDDLFPLIFPSEPAQASGPYVEIIEQPKQRGMRFRYKCEGRSAGSIPGERSTDTTKTHPTIKINGYTGPGTVRISLVTKDPPHRPHPHELVGKDCRDGFYEAELCPDRCIHSFQNLGIQCVKKRDLEQAISQRIQTNNNPFQVPIEEQRGDYDLNAVRLCFQVTVRDPSGRPLRLTPVLSHPIFDNRAPNTAELKICRVNRNSGSCLGGDEIFLLCDKVQKEDIEVYFTGPGWEARGSFSQADVHRQVAIVFRTPPYADPSLQAPVRVSMQLRRPSDRELSEPMEFQYLPDTDDRHRIEEKRKRTYETFKSIMKKSPFNGPTEPRPPPRRIAVPSRGPTSVPKPAPQPYAFSTSLSTINFDEFSPMVLPPGQISNQALALAPSSAPVLAQTMVPSSAMVPSLAQPPAPVPVLAPGPPQSLSAPVPKSTQAGEGTLSEALLHLQFDADEDLGALLGNNTDPGVFTDLASVDNSEFQQLLNQGVAMSHSTAEPMLMEYPEAITRLVTGSQRPPDPAPATLGTSGLPNGLSGDEDFSSIADMDFSALLSQISS

(19) United States
(12) Patent Application Publication
(10) Pub. No.: US 2017/0082610 A1
(43) Pub. Date: Mar. 23, 2017

(54) METHOD FOR PREDICTING TOXICITY OF A COMPOUND BASED ON NUCLEAR FACTOR-kB TRANSLOCATION

(71) Applicant: AGENCY FOR SCIENCE, TECHNOLOGY AND RESEARCH, Singapore (SG)

(72) Inventors: Daniele ZINK, Singapore (SG); Sijing XIONG, Singapore (SG)

(21) Appl. No.: 15/126,577

(22) PCT Filed: Mar. 17, 2015
(86) PCT No.: PCT/SG2015/050039

§ 371 Date: Sep. 15, 2016

(30) Foreign Application Priority Data
Mar. 17, 2014 (SG) 10201400705X

(51) Int. Cl. G01N 33/50

(52) U.S. Cl. CPC G01N 33/5035; G01N 2500/10

(57) ABSTRACT

There is provided a method of screening for toxicity of a compound. The method comprises contacting a test compound with a test population of cells in which nuclear factor (NF)-κB has not been activated prior to the contacting; determining nuclear localization levels of NF-κB in the test population subsequent to the contacting; and comparing nuclear localization levels of NF-κB of a control population that has not been contacted with the test compound. An increase in nuclear localization levels of NF-κB as of the test population relative to the control population is indicative that the test compound injures the cells and/or induces a pro-inflammatory response and thus is toxic to the cell type used in the method.

### CROSS-REFERENCE TO RELATED APPLICATION

This application claims benefit of, and priority from, SG provisional application No. 10201400705X, filed on Mar. 17, 2014, the contents of which are hereby incorporated herein by reference.

### FIELD OF THE INVENTION

The invention relates to in vitro methods of predicting toxicity of a compound, including organ-specific toxicity or tissue-specific toxicity.

### BACKGROUND OF THE INVENTION

The NF-κB family of transcription factors (referred to herein collectively as NF-κB) is a master regulator of inflammation and stress responses in animals and humans [1, 2]. NF-κB is ubiquitously expressed and usually retained in the cytoplasm [3, 4] in an unactivated state.

NF-κB may become activated by a large variety of different stress factors, including cytokines, bacterial toxins, viral products, hypoxia, reactive oxygen species and UV light [3-5]. Upon activation, NF-κB translocates rapidly into the nucleus where it regulates a large number of target genes.

Because of the number and variety of genes targeted by NF-κB, NF-κB activation is associated with a number of diseases, including cancer [1, 6] and inflammatory diseases such as rheumatoid arthritis, atherosclerosis, asthma, multiple sclerosis, inflammatory bowel disease and ulcerative colitis [7]. These diseases affect various organ systems such as lung, bowel, the cardiovascular system or the central nervous system. NF-κB activation is also associated with various forms of renal disease [8, 9]. NF-κB becomes activated in specific cell types that are involved in the disease process or that are affected by certain stressors.

The NF-κB family of transcription factors consist of homo- or heterodimers of different protein subunits. So far, five protein subunits have been identified [5]. Many of the target genes regulated by NF-κB are involved in inflammatory reactions; however, which precise set of genes is targeted depends on the isoform of NF-κB and the subunits involved. The p65 subunit of NF-κB is essential for the activation of the pro-inflammatory interleukins (IL) IL-6 and IL-8 [10-12].

### SUMMARY OF THE INVENTION

There is provided an in vitro assay for the prediction of organ-specific or tissue-specific toxicity of a compound, as reflected by the compound's ability to activate NF-κB and induce its nuclear translocation.

The assay is based on detection of nuclear translocation of NF-κB, for example nuclear translocation of NF-κB subunit p65. Briefly, the selected cell type is first treated with the potentially toxic or nontoxic compound to be screened, and NF-κB translocation is then measured in order to predict the toxicity of a compound and its capability to induce pro-inflammatory NF-κB signalling by activation of NF-κB. The assay is suitable for high content screening (HCS), and in some embodiments HCS may be the preferred method for assessing NF-κB translocation.

The assay may use primary somatic organ- or tissue-specific cells or stem cells, stem-cell derived organ-specific vertebrate cells, germs cells or their precursors, or organ- or tissue-specific established cell lines, i.e. immortal or immortalized cell lines.

In one aspect, the invention provides an in vitro method of screening for toxicity of a compound, the method comprising: contacting a test compound with a test population of cells in which nuclear factor (NF)-κB has not been activated prior to the contacting; determining nuclear localization levels of NF-κB in the test population subsequent to the contacting; and comparing the nuclear localization levels of NF-κB in the test population with nuclear localization levels of NF-κB in a control population of cells that has not been contacted with the test compound; wherein an increase in nuclear localization levels of NF-κB in the test population relative to the control population is indicative that the test compound injures the cells and/or induces a pro-inflammatory response.

The NF-κB assessed in the method may be any isoform or subunit of NF-κB. In some embodiments, the NF-κB is the p65 subunit of NF-κB. In some embodiments, the p65 subunit of NF-κB comprises the sequence set forth in any one of SEQ ID NOs: 1 to 4 or the sequence set forth in SEQ ID NO: 5 or 6.

The cells used in the method, including the cells of the test population and control population may be human cells or may be non-human animal cells.

The cells may comprise stem cells, including for example embryonic stem cells, mesenchymal stem cells, hematopoietic stem cells, induced pluripotent stem cells, tissue-specific stem cells, or organ-specific stem cells.

The cells may comprise somatic cells or cells derived from stem cells.

For example, the cells may comprise tumour cells, germ cells or their precursors, or primary cells. In some embodiments, the cells may comprise liver cells, kidney cells, cardiovascular cells, central nervous system cells, skin cells, lung cells, pancreatic cells, digestive tract cells, eye cells, ear cells, bone marrow cells or blood cells. The cells may comprise renal proximal tubular cells, including for example human primary renal proximal tubular cells.

For example, the cells may comprise cells from an established cell line, including for example HK-2 cells, or LLC-PK1 cells.

For example, the cells may comprise cells derived from stem cells which are differentiated from embryonic stem cells, mesenchymal stem cells, induced pluripotent stem cells or organ/tissue-specific stem cells. In some embodiments, the cells may be at least partially differentiated to resemble liver cells, kidney cells, cardiovascular cells, central nervous system cells, skin cells, lung cells, pancreatic cells, digestive tract cells, germ cells or their precursors, eye cells, ear cells, bone marrow cells or blood cells. In some embodiments, the cells may be renal proximal tubular-like cells.

In the method, the contacting is performed over a period of time, for example, from about 1 hour to about 16 hours, from about 12 hours to about 16 hours, or from about 30 to about 36 hours, or for about 3 days.

The method may further comprise repeating the contacting one or more times, at regular intervals over a total time period of up to 4 weeks, prior to the determining, including wherein the contacting is performed for the same time period for each occurrence of the contacting.

The method may further comprise repeating the contacting followed by the determining one or more times, at regular intervals over a total time period of up to 4 weeks, including wherein the contacting is performed for the same time period for each occurrence of the contacting.

Other aspects and features of the present invention will become apparent to those of ordinary skill in the art upon review of the following description of specific embodiments of the invention in conjunction with the accompanying tables and figures.

### BRIEF DESCRIPTION OF THE DRAWINGS

The figures, which illustrate, by way of example only, embodiments of the present invention, are as follows.

FIG. 1: Schematic drawing comparing prior NF-κB-based assays and embodiments of the method of the invention.

FIG. 2: Overview of a high content screening (HCS) embodiment of the method of the invention, used to detect compound-induced NF-κB translocation.

FIG. 3: (Contains Table 1) Heat Map for 41 different compounds tested for NF-κB translocation.

FIG. 4: (Contains Table 2) EC50 values and highest percentages of positive cells determined for 41 different compounds tested.

FIG. 5: (Contains Table 3) Qualitative analysis of NF-κB translocation for 42 different compounds tested.

FIG. 6: Graphical depiction of sensitivity, specificity and overall concordance with clinical data.

FIG. 7: (Contains Table 4) Heat map for 43 different compounds tested.

FIG. 8: (Contains Table 5) Prediction of drug-induced proximal tubular toxicity using compound-induced NF-κB translocation as an endpoint.

FIG. 9: HPTC1 imaged by HCS (controls).

FIG. 10: HPTC1 imaged by HCS after treatment (at the indicated concentrations) with nephrotoxicants that directly damage the proximal tubule in humans. EC50 and IC50 values were determined for each compound.

FIG. 11: HPTC1 imaged by HCS after treatment (at 1000 μg/mL) with nephrotoxicants that directly damage the proximal tubule in humans.

FIG. 12: HPTC1 imaged by HCS after treatment (at 1000 μg/mL) with compounds that are not directly toxic for proximal tubular cells in humans.

### DETAILED DESCRIPTION

Activation of transcription factor NF-κB results in translocation of ubiquitously expressed NF-κB from the cytoplasm to the nucleus. NF-κB activation is associated with a number of diseases; as a result, there is interest in identifying compounds that modulate or inhibit the activity of activated NF-κB [7, 13, 14].

Thus, assays to detect the impact of a given test compound on NF-κB activity have been previously developed. Such prior assays use an agonist known to induce NF-κB nuclear translocation and thus activation, and then subsequently assess the effect of the test compound on the agonist-induced nuclear localization of NF-κB. Agonists used in such assays typically include tumor necrosis factor (TNF)-α or IL-1. These previously described assays therefore address cell signalling, including signalling pathways that regulate NF-κB, but do not address cytoxicity or the ability to activate NF-κB by the test compound to be screened.

In contrast to previously established NF-κB-based assays that use activators/agonists to activate NF-κB, the current method is based on the hypothesis that a toxic compound imposes stress on a cell, which stress can then activate NF-κB and induce its nuclear translocation. Thus, in the methods described herein, the ability of a test compound (in the absence of any activator or agonist) to induce NF-κB translocation is measured, as a method to determine the toxicity of the compound. As well, the translocation of NF-κB induced by the test compound reflects the potential of the compound to activate NF-κB signalling and induce a pro-inflammatory effect.

In the assay method described herein, the cells are first treated with the potentially toxic test compound that is to be screened for toxicity. NF-κB translocation is then measured in order to determine the toxicity of a compound and its pro-inflammatory potential. Compounds that induce NF-κB translocation are classified as positives and are predicted as being toxic for the specific cell type used.

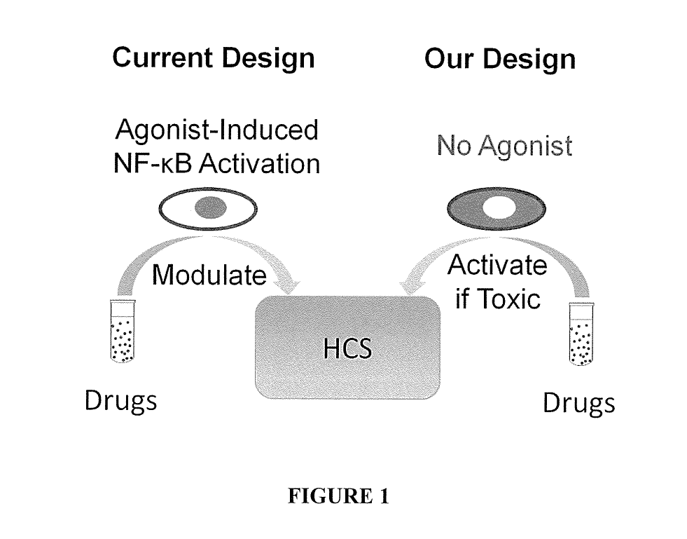

FIG. 1 shows the design of previously known assays used to determine regulation of NF-κB and compares it with the design of the method as described herein.

Previously known NF-κB-based assays (left-hand side of FIG. 1) are designed to detect modulatory effects of a compound on activated NF-κB. Therefore, nuclear translocation of NF-κB is first induced by an agonist (e.g.TNF-α or IL1). The active state is symbolized here by a cell with a light cytoplasm and dark nucleus. The activated samples with increased NF-κB localization in the nucleus are then treated with the compounds to be screened (arrow). Modulatory effects of screened compounds on agonist-induced NF-κB activity are often detected by HCS.

In contrast, in the methods described herein (right-hand side of FIG. 1), the cells are only treated with the compounds to be screened. No agonists are used to induce nuclear translocation of NF-κB. Thus, NF-κB is localized in the cytoplasm and is inactive prior to any contacting with the compounds to be screened (symbolized here by a cell with a dark cytoplasm and light nucleus). Potential NF-κB activation due to toxic cell injury may be detected by HCS.

Notably, activation of NF-κB by a cytokine or another agonist (e.g. TNFα) is not used in the assay method described herein. This feature distinguishes the described assay from the previously known NF-κB-based HCS assays. That is, the assay described herein measures the capability of a compound to induce NF-κB translocation, and not the modulation of NF-κB activity by the test compound subsequent to nuclear translocation of NF-κB induced by another compound.

Thus, in one aspect, there is provided an in vitro method for screening the toxicity of a test compound.

Briefly, the method comprises contacting the test compound with a test population of cells in which NF-κB has not been activated prior to the contacting. The nuclear localization levels of NF-κB are then assessed and compared to the NF-κB nuclear localization levels for a control population that has not been treated with the test compound.

The test compound may be any compound for which toxicity is to be assessed, including a compound for which its ability to activate NF-κB or to induce NF-κB nuclear translocation is not known prior to performing the method. The test compound may be any compound that is expected to come into contact with a subject, including being inhaled by, topically applied to, absorbed by, ingested by, administered to, or implanted into a subject. For example, the test compound may be a pharmaceutical compound, an organic compound, an inorganic compound, a pesticide, a herbicide, an environmental toxin, a fungal toxin, a microbial toxin, a heavy metal-containing compound, an organic solvent, a cleaning agent, a preservative, a food additive, a dietary supplement, a herbal compound, an animal-derived compound, an anti-microbial compound, a cosmetic ingredient, a microparticle or a nanoparticle.

The test compound is contacted with a test population of cells.

The test population of cells, prior to contact with the test compound, has not had activation of NF-κB induced. Thus, most of NF-κB will be located in the cytoplasm of the cells of the test population prior to the contacting, and thus is available to become activated and then translocate to the nucleus, depending on the activity of the test compound during the contacting.

The test population may be any population of cells in which toxicity of the test compound is desired to be assessed.

As used herein, the term “cell”, including when referring to a cell belonging to the control population of cells or the test population of cells, is intended to refer to a single cell as well as a plurality or population of cells, where context allows. Similarly, the term “cells” or “population” of cells is also intended to refer to a single cell, where context allows.

The test population of cells may be composed of any type of cell, including any type of human or non-human animal cell, including mouse cells or rat cells. The cells may be stem cells or may be somatic cells such as primary cells, established cell lines such as immortal or immortalized cells, tumour cells, germs cells or their precursors, as well as cells derived or differentiated from stem cells, including derived or differentiated from induced pluripotent stem cells.

The test population of cells may be a single cell type or may be a mixed population containing two or more different cell types.

Thus, the test population may comprise a population of stem cells. Stem cells include embryonic stem cells, induced pluripotent stem cells, adult stem cells such as mesenchymal stem cells, hematopoietic stem cells or tissue-specific stem cells, or organ-specific stem cells. The stem cells may comprise human embryonic stem cells, including human embryonic stem cells from an existing cell line.

The test population may comprise a population of germs cells or their precursors.

The test population used may comprise somatic cells, cells derived from somatic cells, or cells derived by differentiating stem cells. The cells may comprise primary cells, cells from an established cell line, including an immortalized cell line, or tumour cells. The cells may be differentiated from stem cells, including differentiated from embryonic stem cells, from mesenchymal stem cells, from induced pluripotent stem cells, from tissue-specific stem cells, or from organ-specific stem cells.

Somatic cells and cells derived from somatic cells include primary cells or cells from an established, immortal or immortalized cell line. For example, somatic cells may be any type of organ-specific or tissue-specific cell types, for example and without limitation, liver cells (hepatocytes or other hepatic cell types), kidney cells (glomerular, tubular and other renal cell types), cardiovascular cells (cardiomyocytes, endothelial cells), central nervous system cells (neurons, astrocytes, glia cells), skin cells (keratinocytes, skin fibroblasts and cell types specific for accessory structures: glands and hair follicles), lung cells (airway epithelial cells, alveolar cells), pancreatic cells (beta-cells and other pancreatic cell types), digestive tract cells (different cell types of the stomach and small intestine), cell types specific for reproductive organs, sensory organs (eye- and ear-specific cell types), bone marrow cells or blood cells. In some embodiments, the cells are renal proximal tubular cells, including for example human primary renal proximal tubular cells, HK-2 cells, or LLC-PK1 cells.

Cells derived from stem cells include cells that have been differentiated from embryonic stem cells, from mesenchymal stem cells, from induced pluripotent stem cells, from organ-specific stem cells or from tissue-specific stem cells. For example, cells derived from stem cells include cells at least partially differentiated to resemble liver cells (hepatocytes or other hepatic cell types), kidney cells (glomerular, tubular and other renal cell types), cardiovascular cells (cardiomyocytes, endothelial cells), central nervous system cells (neurons, astrocytes, glia cells), skin cells (keratinocytes, skin fibroblasts and cell types specific for accessory structures: glands and hair follicles), lung cells (airway epithelial cells, alveolar cells), pancreatic cells (beta-cells and other pancreatic cell types), digestive tract cells (different cell types of the stomach and small intestine), cell types specific for reproductive organs including germ cells and their precursors, sensory organs (eye- and ear-specific cell types), bone marrow cells or blood cells. In some embodiments, the cells are renal proximal tubular-like cells.

The use of undifferentiated or partially differentiated stem cells may allow for assessing the embryotoxicity and/or the general toxicity of a compound, without necessarily assessing for tissue specificity or organ specificity.

However, it may be desirable to assess toxicity in a particular cell type, including assessment of tissue-specific toxicity or organ-specific toxicity. NF-κB is ubiquitously expressed in all human and animal cell types and NF-κB activation occurs in many different organ systems in association with injury, disease and inflammation [7]. The use of NF-κB nuclear translocation as endpoint for an in vitro assay for the prediction of organ-specific toxicity is particularly interesting. Previously, it has been difficult to identify suitable endpoints for such in vitro assays. Although endpoints for measuring general cytotoxicity (e.g. cell death, reduced metabolic activity, ATP depletion) are widely used, prediction of organ-specific toxicity with such endpoints has not been successful [16, 17]. Currently, there are no accepted in vitro assays for the prediction of toxic effects on internal human organs (a list of validated and accepted alternative methods is provided at alttox.org/mapp/table-of-validated-and-accepted-alternative-methods/).

In such case, the cells used may be partially or fully differentiated. For example, the cell population may be somatic primary cells specific to a particular type of tissue or organ, cells derived from somatic cells, such as tumour cells or established cell lines, tissue-specific stem cells, organ-specific stem cells, or cells partially or fully differentiated from stem cells, including embryonic stem cells or induced pluripotent stem cells.

Prior to contacting the test compound with the test population of cells, the test population may be first cultured, in accordance with standard tissue culture methods suitable for the type of cell used. Tissue culture conditions and techniques for various cell types are known, for example as described in Molecular Cloning: A Laboratory Manual, 4^{th} ed., by Michael R. Green and Joseph Sambrook, ©2012, Cold Spring Harbor Laboratory Press, and in Ref. [18].

The test population of cells may be cultured in any format, including as a confluent monolayer, a subconfluent monolayer, a confluent epithelium, an organoid culture, a confluent 2D culture, an in vitro tubule, a 3D organoid culture, or a 3D culture including a static 3D culture or a 3D culture grown under microfluidic conditions. As indicated above, the test population may consist of a single cell type or may represent co-cultures of two or more different cell types.

In some embodiments, the cells are grown in a monolayer, such as a confluent or subconfluent monolayer. If automated detection methods are to be used, monolayer cultures are preferred, since 3D cultures involve multiple cell layers which are not suitable for current HCS methods.

For example, cells may be seed at high density (e.g. from about 20 000 cells/cm² to about 50 000 cells/cm²) in multi-well plates. Tissue culture plates made of polystyrene typically do not need any treating with a coating, gel etc. if cells such as immortal cell lines or human primary renal proximal tubular cells (HPTC) are used. If other primary human cell types (e.g. hepatocytes, usually cultivated with collagen I coating) or stem cells are used, the tissue culture plates may require a coating such as Matrigel™ or a synthetic coating. For example, culturing and differentiating hESCs and hiPSCs into HPTC-like cells may involve the use of hESC-qualified Matrigel™ coating on tissue culture plates.

The cells may be cultivated for a suitable period, for example about 1 day or longer, about 3 days or longer, or for about 1 to about 3 days, prior to contacting with the compound in order to provide the cells time to equilibrate, to form a monolayer and to differentiate, if desired. For example, if HPTC are used, a differentiated renal epithelium consisting of a single cell layer (i.e. a simple epithelium) may be formed prior to the contacting with the test compound.

In some embodiments, the cells may be grown in microfluidic bioreactors, including in the form of a confluent or subconfluent monolayer. A microfluidic bioreactor may be useful for long-term cultivation and repeated exposure to a test compound, as described herein. This format may be useful for generating compound concentration gradients within the culture.

As will be appreciated, the same cell type should be used for the test population and control population. The control population should typically be cultured and treated in the same manner as the test population, with the exception of contacting with the test compound. Instead, it may be appropriate to contact the control population with a vehicle control, for example the solution or solvent used to dissolve the test compound but without any included test compound.

In the method, the test compound is then contacted with test population.

The contacting may be done by adding the compound to the culture medium in which the cells are cultured. For example, the compound may be dissolved or dispersed in a liquid vehicle, such as a solvent or solution.

The contacting may be done over a period of time, for example by incubating the compound that is to be tested with the cells in culture.

The contacting may be performed over a period of about 1 minute or longer, about 5 minutes or longer, about 15 minutes or longer, about 1 hour or longer, about 2 hours or longer, about 4 hours or longer, about 8 hours or longer, about 16 hours or longer, about 24 hours or longer, about 36 hours or longer, about 48 hours or longer, about 60 hours or longer, or about 72 hours or longer. The contacting may be performed over a period of from about 15 minutes to about 72 hours, from about 1 hour to about 48 hours, from about 1 hour to about 24 hours, from about 1 hour to about 16 hours, from about 8 hours to about 36 hours, from about 16 hours to about 24 hours, from about 12 hours to about 16 hours, or from about 30 to about 36 hours, or for about 3 days.

The test compound may be left in the cell culture medium, and if the medium requires changing before the total incubation period is complete, then fresh medium that is added may also contain the test compound in order to maintain the contacting.

The concentration of the test compound to be used may be varied, and may depend on the compound that is to be tested.

For example, the test compound may be contacted with the population of cells at a concentration of about 0.001 μg/mL or higher, about 0.01 μg/mL or higher, about 0.1 μg/mL or higher, about 1 μg/mL or higher, about 10 μg/mL or higher, about 100 μg/mL or higher, about 1000 μg/mL or higher, or about 10 000 μg/mL or higher. The test compound may be contacted with the population of cells at a concentration of from about 0.001 μg/ml to about 10 000 μg/ml, from about 0.001 μg/ml to about 1000 μg/ml, from about 0.005 μg/ml to about 5000 μg/ml, from about 0.005 μg/ml to about 1000 μg/ml, from about 0.01 μg/ml to about 1000 μg/ml, or from about 0.01 μg/ml to about 500 μg/ml.

As indicated above, the control population of cells, although not contacted with the test compound, may be contacted with a negative control solution, for example the solvent or solution used to dissolve or disperse the test compound for contacting with the test population (vehicle control).

The contacting may be repeated, including on a periodic basis. For example, the contacting may be performed two or more times, three or more times, four or more times or five or more times over a given period of time, optionally interspersed with periods in which the test population is not contacted with the test compound.

For example, after the first period of contacting is completed, the tissue culture medium may be replaced with fresh medium that contains the test compound. Alternatively, the medium may be replaced with fresh medium that does not contain the test compound, and after a period of time with no contact, the test compound may then again be contacted with the test population of cells.

The contacting thus may be repeated one or more additional times (beyond the first instance of contacting).

For example, the contacting may be repeated, including on a periodic basis, over a period of from about 3 to about 14 days, or from about 3 days to about 4 weeks.

The interval without any contact of test compound (i.e. exposing the cells to fresh medium) may last, for example, from about 16 hours to about 14 days, from about 1 day to about 10 days, from about 1 day to about 3 days, from about 1 day to about 5 days, or from about 2 day to about 3 days, between the periods of contacting.

If repeated, the contacting may be repeated at regular intervals within the total time period. Each episode of contacting may be performed for the same length of time within the total time period.

For example, over a period of about 2 weeks, the test compound may be contacting with the test population for about 8 hours, repeated once every two days.

Again, the control population should be treated in a similar manner as the test population, with the exception that the test compound is excluded from contacting the control population. For example, a vehicle control may be added to the culture medium of the control population, repeated for the same time periods, for the same number of occurrences and over the same total time period, as is done for the contacting of the test compound with the test population.

Once the contacting is completed, the levels of nuclear NF-κB are determined for both the test population and the control population.

Reference to NF-κB includes reference to any family member of the NF-κB transcription factors, including any subunit or isoform of NF-κB. Thus, the NF-κB that is assessed in terms of nuclear localization may be any NF-κB, and may depend on the type of cell used in the test population of cells. Although NF-κB is ubiquitous, the form of NF-κB found in various cell types may differ, and thus the NF-κB that is measured in terms of nuclear localization for the assay should be an appropriate form of NF-κB for the cell type used.

For example, NF-κB that is detected in the method includes the different isoforms or subunits of NF-κB, including for example the p65 subunit of NF-κB.

The NF-κB may comprise, consist essentially of, or consist of, in some embodiments, human NF-κB p65 subunit isoform 1 as set forth in SEQ ID NO: 1:

MDELFPLIFPAEPAQASGPYVEIIEQPKQRGMRFRYKCEGRSAGSIP
GERSTDTTKTHPTIKINGYTGPGTVRISLVTKDPPHRPHPHELVGKDCRD
GFYEAELCPDRCIHSFQNLGIQCVKKRDLEQAISQRIQTNNNPFQVPIEE
QRGDYDLNAVRLCFQVTVRDPSGRPLRLPPVLSHPIFDNRAPNTAELKIC
RVNRNSGSCLGGDEIFLLCDKVQKEDIEVYFTGPGWEARGSFSQADVHRQ
VAIVFRTPPYADPSLQAPVRVSMQLRRPSDRELSEPMEFQYLPDTDDRHR
IEEKRKRTYETFKSIMKKSPFSGPTDPRPPPRRIAVPSRSSASVPKPAPQ
PYPFTSSLSTINYDEFPTMVFPSGQISQASALAPAPPQVLPQAPAPAPAP
AMVSALAQAPAPVPVLAPGPPQAVAPPAPKPTQAGEGTLSEALLQLQFDD
EDLGALLGNSTDPAVFTDLASVDNSEFQQLLNQGIPVAPHTTEPMLMEYP
EAITRLVTGAQRPPDPAPAPLGAPGLPNGLLSGDEDFSSIADMDFSALLS
QISS

The NF-κB may comprise, consist essentially of, or consist of, in some embodiments, human NF-κB p65 subunit isoform 2 as set forth in SEQ ID NO: 2:

MDELFPLIFPAEPAQASGPYVEIIEQPKQRGMRFRYKCEGRSAGSIP
GERSTDTTKTHPTIKINGYTGPGTVRISLVTKDPPHRPHPHELVGKDCRD
GFYEAELCPDRCIHSFQNLGIQCVKKRDLEQAISQRIQTNNNPFQEEQRG
DYDLNAVRLCFQVTVRDPSGRPLRLPPVLSHPIFDNRAPNTAELKICRVN
RNSGSCLGGDEIFLLCDKVQKEDIEVYFTGPGWEARGSFSQADVHRQVAI
VFRTPPYADPSLQAPVRVSMQLRRPSDRELSEPMEFQYLPDTDDRHRIEE
KRKRTYETFKSIMKKSPFSGPTDPRPPPRRIAVPSRSSASVPKPAPQPYP
FTSSLSTINYDEFPTMVFPSGQISQASALAPAPPQVLPQAPAPAPAPAMV
SALAQAPAPVPVLAPGPPQAVAPPAPKPTQAGEGTLSEALLQLQFDDEDL
GALLGNSTDPAVFTDLASVDNSEFQQLLNQGIPVAPHTTEPMLMEYPEAI
TRLVTGAQRPPDPAPAPLGAPGLPNGLLSGDEDFSSIADMDFSALLSQIS
S

The NF-κB may comprise, consist essentially of, or consist of, in some embodiments, human NF-κB p65 subunit isoform 3 as set forth in SEQ ID NO: 3:

MDELFPLIFPAEPAQASGPYVEIIEQPKQRGMRFRYKCEGRSAGSIP
GERSTDTTKTHPTIKINGYTGPGTVRISLVTKDPPHRPHPHELVGKDCRD
GFYEAELCPDRCIHSFQNLGIQCVKKRDLEQAISQRIQTNNNPFQVPIEE
QRGDYDLNAVRLCFQVTVRDPSGRPLRLPPVLSHPIFDNRAPNTAELKIC
RVNRNSGSCLGGDEIFLLCDKVQKEDIEVYFTGPGWEARGSFSQADVHRQ
VAIVFRTPPYADPSLQAPVRVSMQLRRPSDRELSEPMEFQYLPDTDDRHR
IEEKRKRTYETFKSIMKKSPFSGPTDPRPPPRRIAVPSRSSASVPKPAPG
PPQAVAPPAPKPTQAGEGTLSEALLQLQFDDEDLGALLGNSTDPAVFTDL
ASVDNSEFQQLLNQGIPVAPHTTEPMLMEYPEAITRLVTGAQRPPDPAPA
PLGAPGLPNGLLSGDEDFSSIADMDFSALLSQISS

The NF-κB may comprise, consist essentially of, or consist of, in some embodiments, human NF-κB p65 subunit isoform 4 as set forth in SEQ ID NO: 4:

MDELFPLIFPAEPAQASGPYVEIIEQPKQRGMRFRYKCEGRSAGSIP
GERSTDTTKTHPTIKINGYTGPGTVRISLVTKDPPHRPHPHELVGKDCRD
GFYEAELCPDRCIHSFQNLGIQCVKKRDLEQAISQRIQTNNNPFQVPIEE
QRGDYDLNAVRLCFQVTVRDPSGRPLRLPPVLSHPIFDNRAPNTAELKIC
RVNRNSGSCLGGDEIFLLCDKVQKEDIEVYFTGPGWEARGSFSQADVHRQ
VAIVFRTPPYADPSLQAPVRVSMQLRRPSDRELSEPMEFQYLPDTDDRHR
IEEKRKRTYETFKSIMKKSPFSGPTDPRPPPRRIAVPSRSSASVPKPAPQ
PYPFTSSLSTINYDEFPTMVFPSGQISQASALAPAPPQVLPQAPAPAPAP
AMVSALAQRPPDPAPAPLGAPGLPNGLLSGDEDFSSIADMDFSALLSQIS
S

The NF-κB may comprise, consist essentially of, or consist of, in some embodiments, mouse NF-κB p65 subunit as set forth in SEQ ID NO: 5:

MDDLFPLIFPSEPAQASGPYVEIIEQPKQRGMRFRYKCEGRSAGSIP
GERSTDTTKTHPTIKINGYTGPGTVRISLVTKDPPHRPHPHELVGKDCRD
GYYEADLCPDRSIHSFQNLGIQCVKKRDLEQAISQRIQTNNNPFHVPIEE
QRGDYDLNAVRLCFQVTVRDPAGRPLLLTPVLSHPIFDNRAPNTAELKIC
RVNRNSGSCLGGDEIFLLCDKVQKEDIEVYFTGPGWEARGSFSQADVHRQ
VAIVFRTPPYADPSLQAPVRVSMQLRRPSDRELSEPMEFQYLPDTDDRHR
IEEKRKRTYETFKSIMKKSPFNGPTEPRPPTRRIAVPTRNSTSVPKPAPQ
PYTFPASLSTINFDEFSPMLLPSGQISNQALALAPSSAPVLAQTMVPSSA
MVPLAQPPAPAPVLTPGPPQSLSAPVPKSTQAGEGTLSEALLHLQFDADE
DLGALLGNSTDPGVFTDLASVDNSEFQQLLNQGVSMSHSTAEPMLMEYPE
AITRLVTGSQRPPDPAPTPLGTSGLPNGLSGDEDFSSIADMDFSALLSQI
SS

The NF-κB may comprise, consist essentially of, or consist of, in some embodiments, rat NF-κB p65 subunit as set forth in SEQ ID NO: 6:

MDDLFPLIFPSEPAQASGPYVEIIEQPKQRGMRFRYKCEGRSAGSIP
GERSTDTTKTHPTIKINGYTGPGTVRISLVTKDPPHRPHPHELVGKDCRD
GFYEAELCPDRCIHSFQNLGIQCVKKRDLEQAISQRIQTNNNPFQVPIEE
QRGDYDLNAVRLCFQVTVRDPSGRPLRLTPVLSHPIFDNRAPNTAELKIC
RVNRNSGSCLGGDEIFLLCDKVQKEDIEVYFTGPGWEARGSFSQADVHRQ
VAIVFRTPPYADPSLQAPVRVSMQLRRPSDRELSEPMEFQYLPDTDDRHR
IEEKRKRTYETFKSIMKKSPFNGPTEPRPPPRRIAVPSRGPTSVPKPAPQ
PYAFSTSLSTINFDEFSPMVLPPGQISNQALALAPSSAPVLAQTMVPSSA
MVPSLAQPPAPVPVLAPGPPQSLSAPVPKSTQAGEGTLSEALLHLQFDAD
EDLGALLGNNTDPGVFTDLASVDNSEFQQLLNQGVAMSHSTAEPMLMEYP
EAITRLVTGSQRPPDPAPATLGTSGLPNGLSGDEDFSSIADMDFSALLSQ
ISS

In some embodiments, the NF-κB comprises, consists or consists essentially of, a protein having about 75% or greater, about 80% or greater, about 85% or greater, about 90% or greater, about 95% or greater, about 96% or greater, about 97% or greater, about 98% or greater, or about 99% or greater sequence identity with any one of SEQ ID NO: 1 to SEQ ID NO: 6, while still possessing the function of NF-κB.

As used herein, “consists essentially of” or “consisting essentially of” means that the protein sequence includes one or more amino acid residues, for example one or more, two or more, three or more, four or more, five or more, six or more, seven or more, eight or more, nine or more, ten or more, one to ten, or one to five, at one or both ends of the sequence, and/or within the sequence, but that the additional amino acids do not materially affect the function of the protein.

Thus, following contacting with the test compound, the level of NF-κB in the nucleus of the test population is determined. This may be done in terms of absolute value of NF-κB levels in the nucleus, or it may be achieved by comparing relative amounts of NF-κB between the nucleus and the cytoplasm. For example, determining the nuclear localization levels of NF-κB in the test population may be achieved by determining the cytoplasmic/nuclear ratio of NF-κB levels within the test population.

Nuclear NF-κB levels may be determined using imaging techniques combined with quantitative image analysis analysis. Various methods for locating, imaging and quantifying molecules within cells are known, including immunostaining techniques using fluorescently labelled antibodies directed against NF-κB, detection methods using primary antibodies directed against NF-κB and labelled secondary antibodies, or imaging of fluorescent NF-κB fusion proteins expressed in a transiently or stably transfected cell line.

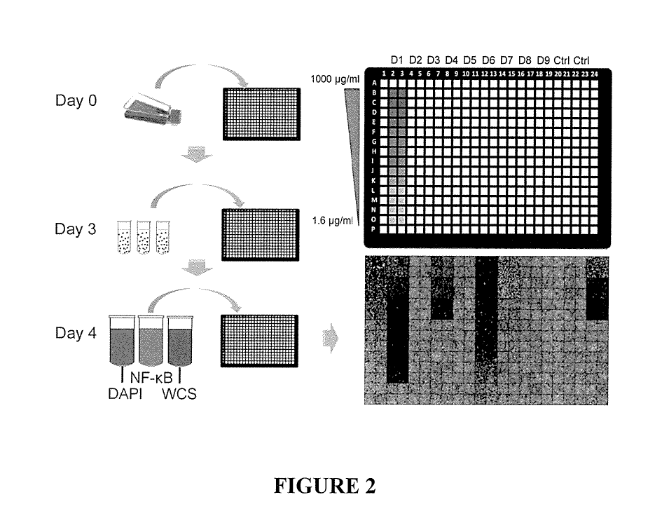

The determining of levels of NF-κB in the nucleus and optionally cytoplasm may comprise automated imaging and image analysis techniques, including high content screening (HCS) techniques. HCS techniques are known, and involve automatic imaging of large numbers of cells that have been seeded or cultured in multi-well plates, followed by subsequent quantitative image analysis. An overview of an embodiment of the method using an HCS procedure is depicted in FIG. 2. Frequently used synonyms for HCS include high-throughput screening or high-content imaging. In fact, HCS assays based on detecting and quantifying NF-κB translocation have been established [13-15]. For example, specific subunits of NF-κB, including the p65 subunit, have been detected by immunostaining. Alternatively, fusion protein techniques have been used, in which a subunit of NF-κB is fused to a fluorescent reporter protein. Such HCS assays have been established with human and animal transformed and cancer cell lines.

Thus, the use of computer-assisted detection techniques may assist in examining the NF-κB localization levels in the nucleus and cytoplasm. The assay may be performed using robotic or automated devices or microfluidic devices in order to increase speed.

In the method, once the NF-κB localization levels have been assessed for both the test population and the control population, then the NF-κB localization levels obtained for the test population are compared with the value obtained for the control population of cells when cultured under the same conditions minus the contact with the test compound.

An increase in nuclear localization levels of NF-κB in the test population relative to the control population is indicative that the test compound injures the cells and/or induces a pro-inflammatory response, meaning the test compound is toxic for that cell type. Thus, for example, a decrease in the ratio of cytoplasm/nuclear localization levels (or an increase in the ratio of nuclear/cytoplasm localization levels) of NF-κB in the test population relative to the control population is indicative that the test compound injures the cells and/or induces a pro-inflammatory response, and thus is toxic for that cell type.

It may be desirable to set a threshold level for detection of the NF-κB localization levels. This may be done by using positive and negative control populations treated with compounds that are known to be non-toxic for the type of cell being used, a set of compounds that are known to be generally toxic, and if tissue-specific or organ-specific toxicity is being assessed, a set of compounds that are known to be specifically toxic to the cell type used. Positive and negative controls may also be used to determine overall assay performance. The numbers of true positives, false positives, true negatives and false negatives may be determined by comparing the results of the in vitro assay with in vivo data. Subsequently, the major performance metrics (sensitivity, specificity, balanced accuracy, positive predictive value, negative predictive value and the area under the curve (AUC) of the receiver operating characteristic curves) can be determined.

In addition, a threshold value may be determined in order to decide whether a test result is positive or negative. The threshold value relates to the fold increase in NF-κB nuclear localization relative to the vehicle control.

For example, a test result may be positive if the increase in the nuclear localization level of NF-κB, is similar to or greater than the threshold value. In some cases, the nuclear localization levels of NF-κB may be assessed by determining the cytoplasmic/nuclear ratio of NF-κB. It will be appreciated that a decrease in the cytoplasmic/nuclear ratio of NF-κB corresponds to an increase in nuclear localization of NF-κB. Thus, if the cytoplasmic/nuclear ratio is used, a positive value is typically lower than the threshold value. Optimal threshold values can be determined by testing broader ranges of threshold values. Actual threshold values may vary depending on the type of cell used and culture and contacting conditions used.

Alternatively, automated classification methods can be used for the results. This can be done, for instance, by using machine learning algorithms such as support vector machine or random forest.

For any given test compound a dose response curve may be calculated by testing the compound at increasing concentrations and comparing the results for each concentration to results for a control population. In this way, an EC₅₀ value may be obtained for test compounds that are found to be toxic in the method.

The present methods and uses are further exemplified by way of the following non-limiting examples.

### EXAMPLES

We developed an HCS-based in vitro model for the prediction of renal proximal tubular (PT) toxicity in humans that uses nuclear translocation of NF-κB (e.g. subunit p65) as endpoint.

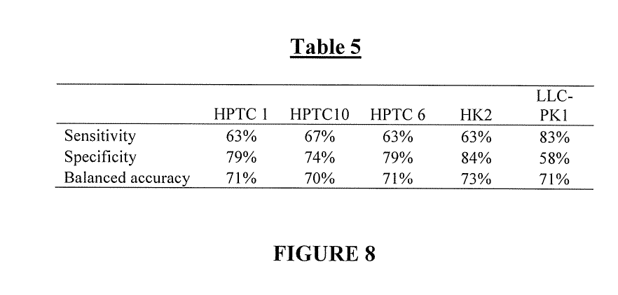

As set out below in the following Examples 2 and 3, using human primary renal proximal tubular cells (HPTC) or proximal tubular cell lines (HK-2 and LLC-PK1), the balanced accuracy of the model was found to be at least 70% (Table 5 (FIG. 8)).

The results obtained as described below demonstrate that nuclear translocation of NF-κB can be used as endpoint in in vitro assays for the prediction of organ-specific toxicity and are in agreement with our previous findings. Previously, we have shown that measuring the up-regulation of IL-6 and IL-8 results in excellent predictivity with respect to the PT toxicity of compounds, using HPTC, HK-2 and LLC-PK1 cells [18] or HPTC-like cells derived from human embryonic [20] or induced pluripotent stem cells (Kandasamy, Chuah et al., manuscript submitted). IL-6 and IL-8 are target genes of NF-κB and are up-regulated by p65 [10-12]. It is important to note that the IL-6/IL-8 based assay [18, 20] is based on qPCR and is not compatible with HCS. Thus, the IL-6/IL-8-based assay has much lower throughput and is much more expensive than the NF-κB-based assay when combined with HCS.

### Example 1

An overview of the HCS method for detecting compound-induced NF-κB translocation is provided in FIG. 2. The figure shows a flow diagram (straight arrows) of the cell seeding and treatment procedures.

As depicted in the embodiment shown in FIG. 2, cells were seeded on day 0 into multi-well plates (in the example shown in FIG. 2, 384-well plates were used). After seeding, cells were cultivated to allow the formation of a confluent monolayer. Compound treatment was performed on day 3 overnight for 16 hours, and cells were then fixed on day 4 and stained, for example using 4′,6-diamidino-2-phenylindole (DAPI, cell nuclei) and whole cell stain (WCS) for the visualization of the overall cell. NF-κB p65 was detected by immunostaining. After staining, the plate was imaged by HCS. In some experiments described in Examples 2 and 3 below, F-actin was also detected.

The lower right portion of FIG. 2 shows the HCS results for one plate and one multi-channel image per well is shown (note that 9 multi-channel images per well were captured; wells at the edge of the plate are not shown and were not used to avoid edge effects;). An outline of the plate design is shown on the top right of the figure.

Each compound (D1-D9) was applied to 2 columns (4 wells (replicates) per concentration) with increasing concentrations (1.6 μg/mL-1000 μg/mL) from bottom to top. The positions of the controls (Ctrl) are indicated. Wells depleted in cells due to cell death appeared dark (right, bottom).

### Example 2

### Methods

Compounds and Definitions:

The same set of 41 compounds as described previously (Refs. [18] and [20]) was used. Simvastatin was included as 42nd compound, but was excluded from most analyses due to its general high cytotoxicity. Compounds 1-22 (group 1) are nephrotoxic in humans and are toxic for proximal tubular (PT) cells. Compounds 23-33 (group 2) are nephrotoxic in humans, but are not toxic for PT cells. Compounds 34-41 (group 3) are not nephrotoxic in humans. True positives (TP) were defined as group 1 compounds that gave a positive test result in vitro. True negatives (TN) were defined as group 2 and group 3 compounds, that gave a negative test result in vitro. Sensitivity was defined as number of TP/22 (group 1) compounds. Specificity was defined as number of TN/19 (group 2+3) compounds.

Cell Culture and Compound Treatment:

Commercial HPTC (ATCC, PCS-400-010) were cultured in Renal Cell Basal Medium (ATCC, PCS-400-030) supplemented with the Renal Epithelial Cell Growth Kit (ATCC, PCS-400-040). Cells at passage 4 or passage 5 were seeded into 96-well Falcon black plates at a density of 50,000 cells/cm². The cells were cultured for 3 days before drug treatment overnight (16 hours). All compounds were screened at concentrations of 1000, 500, 250, 125, 63, 31, 16 and 1.6 μg/mL. Arsenic(III)oxide at 25 μg/mL was used as positive control. Negative controls (cells were not treated with any compounds or vehicles) and vehicle controls were included on each plate. 3 replicates were included for each data point.

Immunostaining:

After treatment with the compounds overnight the cells were fixed using 3.7% formaldehyde in phosphate-buffered saline (PBS). Cells were blocked for 1 hour with PBS containing 5% bovine serum albumin (BSA) and 0.2% Triton X-100. An anti-NF-κB p65 antibody (1:100, sc-372; Santa Cruz Biotechnology, Santa Cruz, Calif., USA) was incubated with the samples at room temperature for 1.5 hours. The secondary antibody was an Alexa Fluor 488 Goat Anti-Rabbit IgG (H+L) (A11008; 1:200; Molecular Probes, Life Technologies, Singapore). Samples were incubated with the secondary antibody and rhodamine phalloidin (Molecular Probes, R415) at room temperature for 1 h (F-actin patterns detected with rhodamine phalloidine were also assessed, but this was unrelated to NF-κB). Finally, 4′,6-diamidino-2-phenylindole (DAPI) (4 ng/mL) was used to stain cell nuclei.

HCS and Image Analysis:

HCS was performed with the ImageXpress^{MICRO} System Version 2.0 (Molecular Devices, Wokingham, UK). 4 images per well were captured. MetaXpress™ image analysis software (version 2.0; Molecular Devices) was used to quantify the percentages of positive cells. A cell was defined as positive for NF-κB translocation, when the nuclear area (defined by DAPI staining) had an equal or higher median intensity of green fluorescence than a defined area of the cytosol. This cytosolic area consisted of a ring of 2 μm thickness around nuclear area.

Results

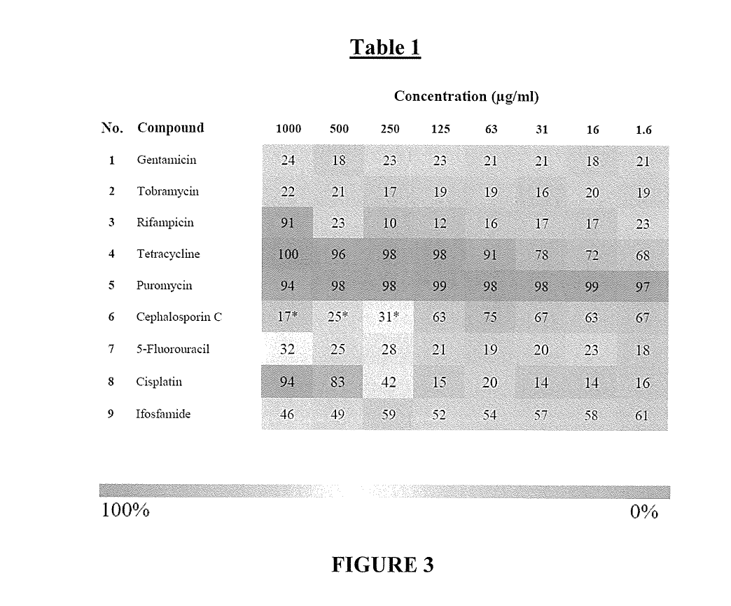

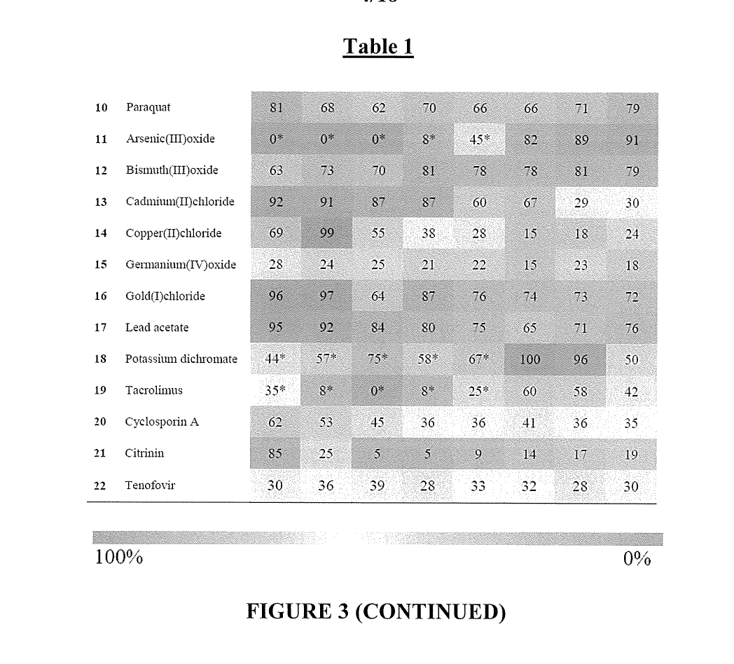

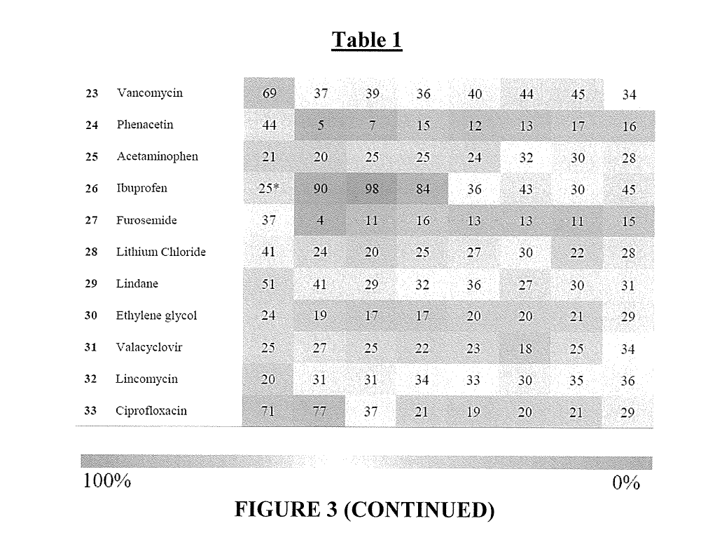

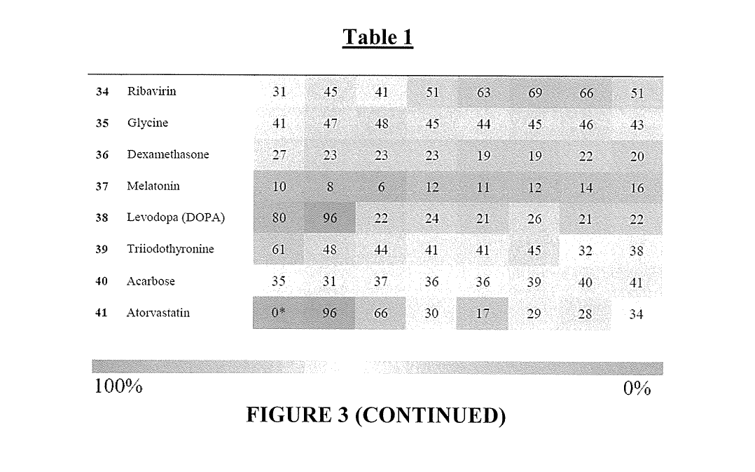

Table 1 (FIG. 3) contains a heat map for 41 different test compounds used in the assay; HPTC were treated with the indicated 41 drugs for 16 hours. The following drug concentrations were used: 1000, 500, 250, 125, 63, 31, 16 and 1.6 μg/mL. The cells were imaged by HCS and the HCS data set is identical with the data set used in Table 3 below, with the exception that drug No. 42 (Simvastatin) is not included in Table 1.

Image analysis was performed as outlined in the Methods section. The number in each box indicates percentage of cells (average of 3 replicates with 4 images per replicate) that were positive and displayed nuclear translocation of NF-κB. Asterisks label cases where cell numbers were low due to cell death.

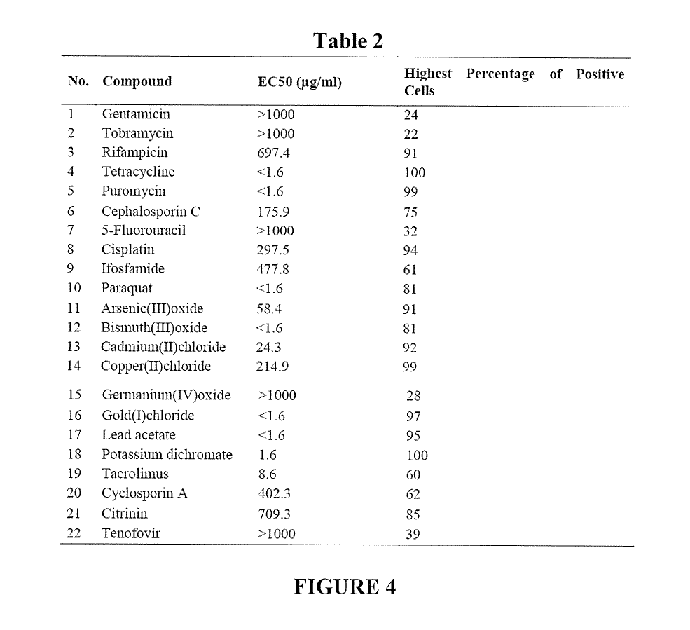

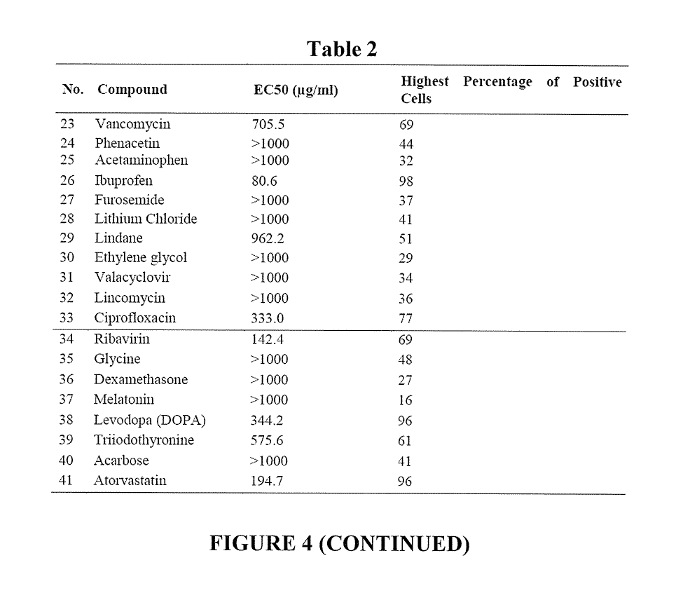

Table 2 (FIG. 4) provides EC50 values derived from the results shown in Table 1 and summarizes the highest percentages of positive cells at any given concentration of a compound within the concentration range tested (highest value in each row in Table 1).

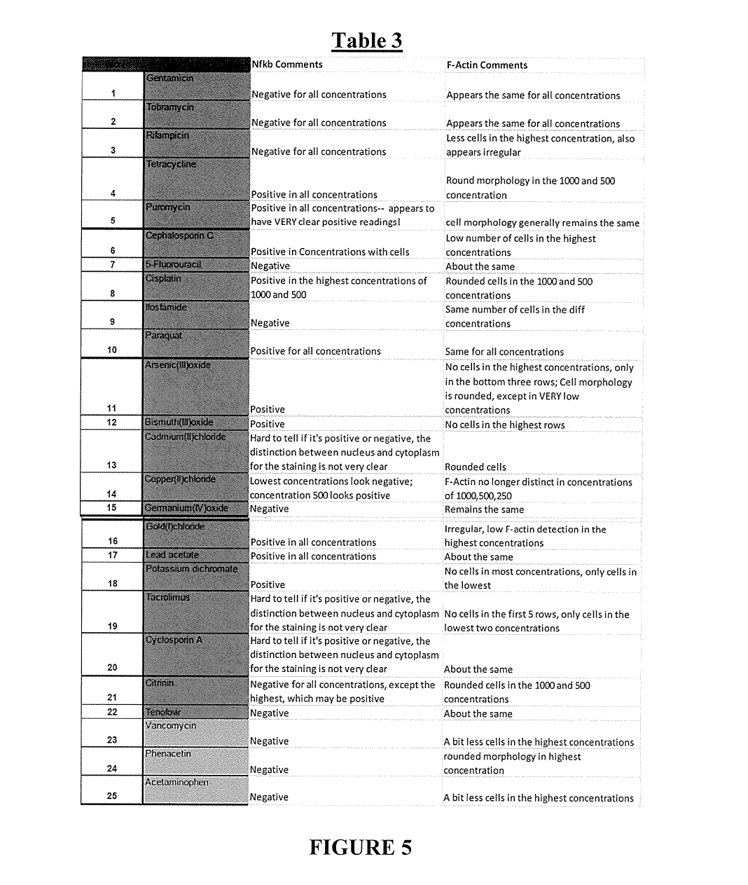

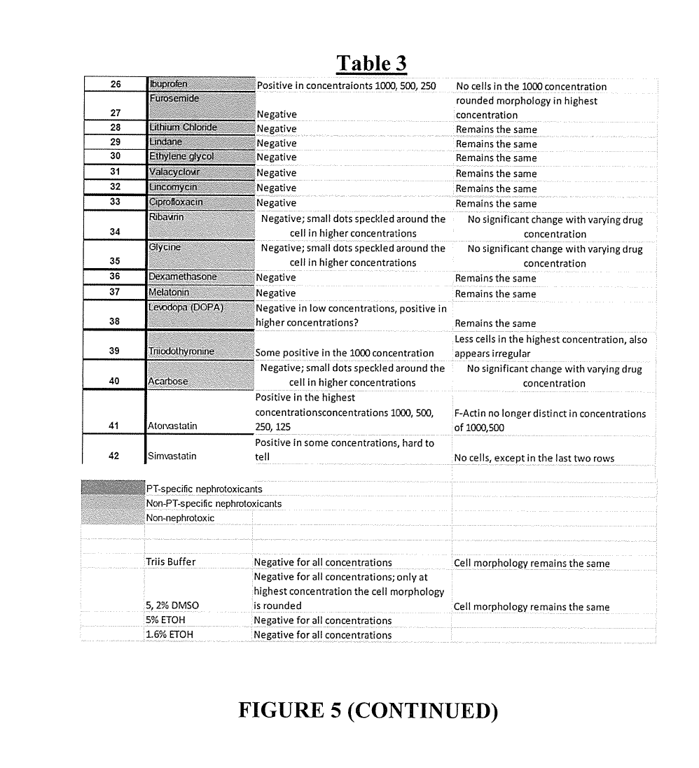

The content of qualitative analysis of NF-κB translocation is shown in Table 3 (FIG. 5), as determined by visual inspection. The HCS work from which the results summarized in Table 3 were derived was performed during the period of Jun. 20, 2013 to Jul. 5, 2013. The screen was performed with 96-well plates, which were seeded with cells, treated with drugs, immunostained and imaged by HCS. During the screen results obtained with individual compounds were noted, which were re-evaluated when the whole screen had been finished on Jul. 5, 2013 and compiled in Table 3. For instance, positive results were obtained with paraquat, arsenic (III) oxide, bismuth (III) oxide, gold (I) chloride, lead acetate, potassium dichromate and tetracycline.

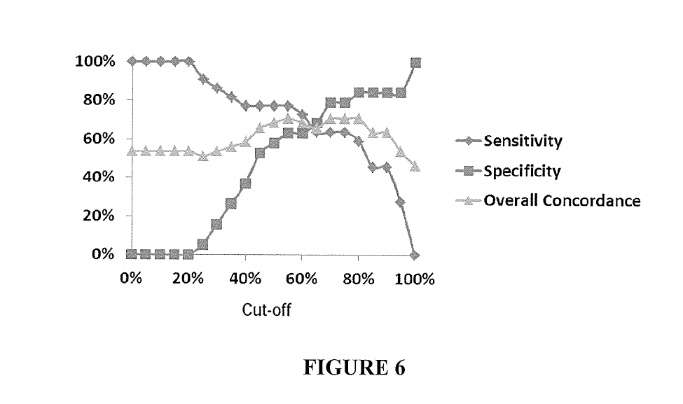

FIG. 6 shows the percentages (y-axis) of sensitivity, specificity and overall concordance with clinical data. The cut-off value (x-axis) refers to the percentage of positive cells. For instance, at a cut-off value of 70% all results were classified as positive where NF-κB translocation was induced in 70% of the cells or more (at any concentration within the range tested, Table 2, right-hand column). A cut-off value of 70% resulted in an overall concordance with human clinical data and balanced accuracy (mean of sensitivity+specificity) of >70%.

### Example 3

### Methods

Compounds and Definitions:

A set of 43 compounds was used, comprising 38 of the set of 41 compounds from Example 2 (excluding tobramycin, ifosfamide, atorvastatin) and 5 new compounds (cephaloridine, cephalothin, metformin hydrochloride, aristolochic acid and ochratoxin A). Compounds 1-24 (group 1) are nephrotoxic in humans and are toxic for proximal tubular (PT) cells. Compounds 25-43 (group 2) are either not nephrotoxic in humans, or are nephrotoxic in humans, but are not directly toxic for PT cells. True positives (TP) were defined as group 1 compounds that gave a positive test result in vitro. True negatives (TN) were defined as group 2 compounds that gave a negative test result in vitro. Sensitivity was defined as (number of TP)/24 (group 1) compounds. Specificity was defined as (number of TN)/19 (group 2) compounds. Balanced accuracy is the mean value of sensitivity and specificity.

Cell Culture and Compound Treatment:

Three different batches of HPTC were used. Commercial HPTC, Lot. No. 58488852 (HPTC 1) and Lot. No. 61247356 (HPTC10), were bought from the American Type Culture Collection (ATCC, Manassas, Va., USA). HPTC6 were isolated from a nephrectomy sample obtained from the National University Health System (NUHS, Singapore). Only normal tissue without pathological changes was used, which was selected by a pathologist. All three batches of HPTC were cultured in ATCC Renal Epithelial Cell Basal Medium (ATCC, Cat. No. PCS-400-030) supplemented with Renal Epithelial Cell Growth Kit (ATCC, Cat. No. PCS-400-040) and 1% Penicillin Streptomycin (Gibco, Cat. No. 15140-122). Only passage (P) 4 and P5 of HPTC were used. HK-2 and LLC-PK1cells were purchased from ATCC. These two cell lines were maintained in Dulbecco's Modified Eagle Medium (DMEM) supplemented with 10% fetal bovine serum (FBS) and 1% Penicillin Streptomycin. Approval for the work with human kidney samples (DSRB-E/11/143) and cell types (NUS-IRB Ref. Code: 09-148E) had been obtained. All cells were incubated in a humidified atmosphere at 37° C. and 5% CO₂.

Cells were seeded into 384-well black plates with transparent bottom (Greiner, Cat. No. 781091). HPTC1 and HPTC6 were seeded at 50,000 cells/cm²; HPTC10 were seeded at 100,000 cells/cm²; HK-2 cells were seeded at 20,000 cells/cm² and LLC-PK1 cells were seeded at 10,000 cells/cm² to adjust for the different growth kinetics of the different cell types and batches used. All cells were cultivated for 3 days to achieve the formation of a differentiated simple epithelium before drug treatment overnight (16 hours). The concentrations of the 43 drugs screened were 1000, 500, 250, 125, 63, 16 and 1.6 μg/mL. Puromycin at 100 μg/mL and TNF-α at 100 ng/mL were used as positive controls. Negative controls included cells that were left untreated (no vehicle, no compound) and cells treated with 100 μg/mL dexamethasone. Vehicle controls were also included on each plate. 4 replicates were tested for each compound and concentration.

Immunostaining:

After treatment with the compounds overnight the cells were fixed using 3.7% formaldehyde in phosphate-buffered saline (PBS). Cells were blocked for 1 hour with PBS containing 5% bovine serum albumin (BSA) and 0.2% Triton X-100. Samples were incubated with an anti-NF-κB p65 antibody (Abcam, Cat. No. ab16502) overnight at 4° C. Alexa Fluor 488 goat anti-rabbit IgG (H+L) (Invitrogen, Cat. No. A11008) was used as secondary antibody. Finally, the cells were counterstained with DAPI (Merck, Cat. No. 268298), rhodamine phalloidin (Invitrogen, Cat. No. R415) and whole cell stain red (Cellomics, Cat. No. 8403401). Detection of F-actin with rhodamine phalloidin was unrelated to the work on NF-κB.

HCS and Image Analysis:

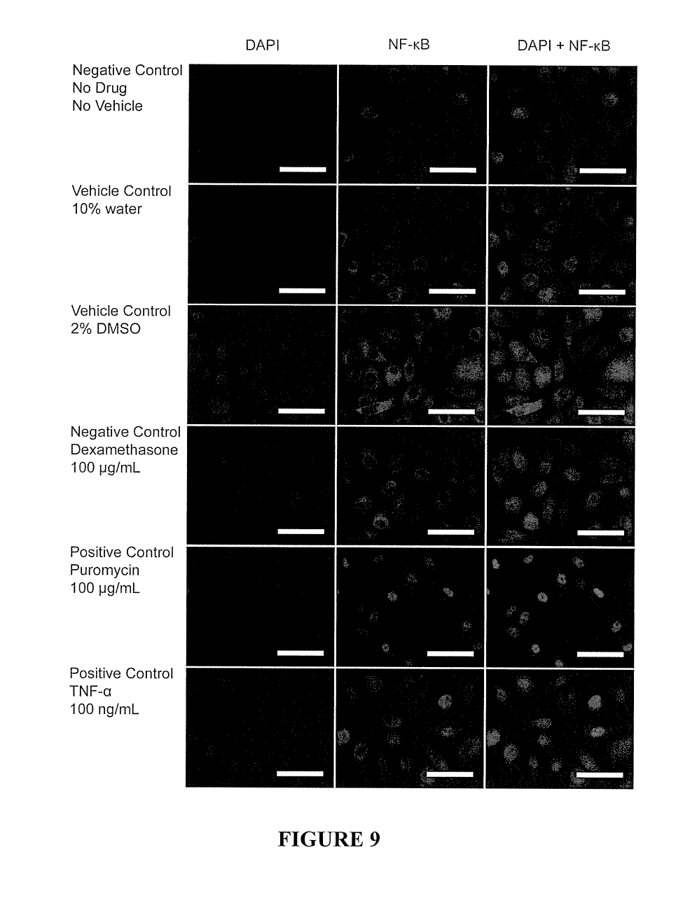

Image acquisition by HCS was performed with the same equipment and software as described in Examples 1 and 2. Four different channels were used for detecting DAPI (cell nuclei), Alexa Fluor 488 (NF-κB; for better perception of the staining patterns NF-κB staining is shown in red in FIGS. 9-12), rhodamine phalloidin (F-actin) and Cy5 (whole cell stain red). 9 sites per well were imaged (all 4 channels). The images were automatically analysed by using MetaXpress™ image analysis software version 2.0 using the Translocation-Enhanced Module [19]. The DAPI stained area was used to define inner nuclear area (nucleus) and outer nuclear area (cytoplasm surrounding the nucleus). An area within 2 μm from the edge of the DAPI stained area was defined as inner nuclear area (nucleus). A ring from 0.1 μm to 2 μm from the outer edge of the DAPI stained area was defined as outer nuclear area (cytoplasm). The intensity ratios of the outer nuclear area versus the inner nuclear area were automatically quantified by the MetaXpress™ image analysis software (version 2.0). The final quantitative results for each data point were the average results from 36 images (4 replicates (wells) with 9 multi-channel images per well).

Results

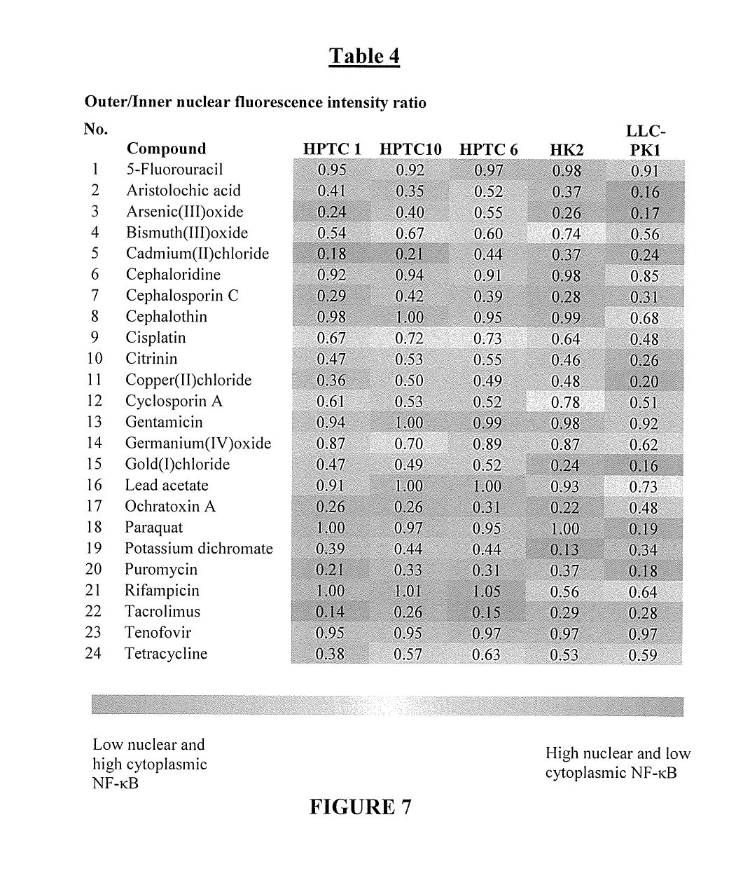

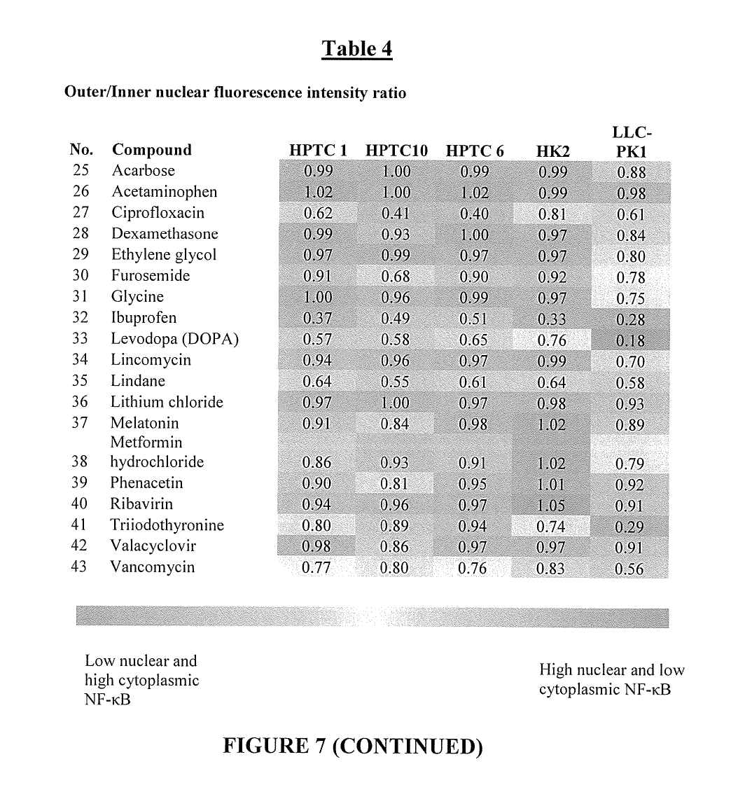

Table 4 (FIG. 7) depicts a heat map. Three batches of HPTC and two cell lines (HK-2 and LLC-PK1) were treated with the indicated 43 compounds for 16 hours. The following compound concentrations were used: 1000, 500, 250, 125, 63, 16 and 1.6 μg/mL. The cells were imaged by HCS using the ImageXpress^{MICRO} system. The images were automatically analysed by the MetaXpress™ image analysis software version 2.0 as described in the Methods section. The average intensity ratios of the outer nuclear area (cytoplasm) versus the inner nuclear area (nucleus) were determined for each compound at each concentration tested. The number in each box shows the lowest intensity ratio obtained for each compound with respect to the whole range of concentrations tested (7 values were obtained for the 7 concentrations tested and the lowest value was selected and is shown in Table 4).

The results shown in Table 4 were classified as positive or negative by using a cut-off value of 0.75 (all results at or below 0.75 classified as positive). Table 5 (FIG. 8) provides the results obtained for sensitivity, specificity and balanced accuracy by using this cut-off value. 3 different batches of HPTC and 2 cell lines (HK-2 and LLC-PK1) were used.

As shown in FIG. 9, HPTC1 were imaged by HCS in the untreated state (top) or after treatment with the indicated control compounds. DAPI stained cell nuclei (blue, left) or NF-κB-staining (red, middle) are shown. The merged images are shown on the right. The nuclear area was depleted of NF-κB in untreated cells, vehicle controls and negative controls (“dark whole” in the center of the cells stained for NF-κB (red, middle). Positive control samples (puromycin at 100 μm/mL and TNF-α at 100 ng/mL) displayed nuclear translocation of NF-κB (increased intensity of NF-κB in the DAPI-positive nuclear area). Scale bars: 50 μm.

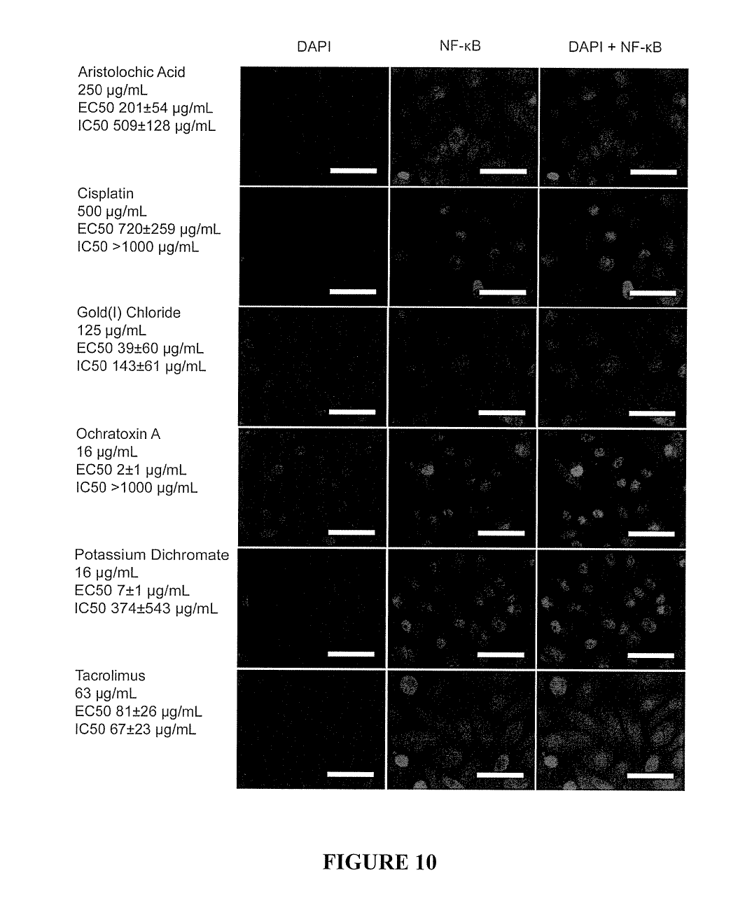

As shown in FIG. 10, HPTC1 were imaged by HCS after treatment with the indicated compounds. DAPI stained cell nuclei (blue, left) or NF-κB-staining (red, middle) is shown. The merged images are displayed on the right. All compounds were nephrotoxicants that are directly toxic for proximal tubular cells in humans. The concentrations used in the selected cases shown are provided below the compound name (every compound was tested at the full range of concentrations, but not all of the images can be shown here). The concentrations selected here were the lowest concentrations of a compound where NF-κB translocation from cytosol to nucleus was observed. Based on the HCS results obtained with the full range of concentrations tested, EC50 (compound concentration where nuclear translocation of NF-κB occurred in 50% of cells) and IC50 (compound concentration where cell death occurred in 50% of cells; results based on counts of cell nuclei) values were calculated. An IC50 value of >1000 μg/mL is indicated where >50% of cells were still present at the highest compound concentration of 1000 μg/mL. Based on such high IC50 values of >1000 μg/mL respective compounds would have been predicted as not being toxic for proximal tubular cells in humans if cell death would have been used as endpoint to predict toxicity (all compounds included in this figure are toxic for this cell type). In contrast, in all cases nuclear translocation of NF-κB was observed in >50% of cells within the concentration range tested and all compounds would be predicted as being toxic based on these results. These results show that the sensitivity is higher when NF-κB translocation is used as endpoint compared to cell death. This is also reflected by the fact that the EC50 values are lower than the IC50 values (with the exception of Tacrolimus, where both values are relatively low). Scale bars: 50 μm.

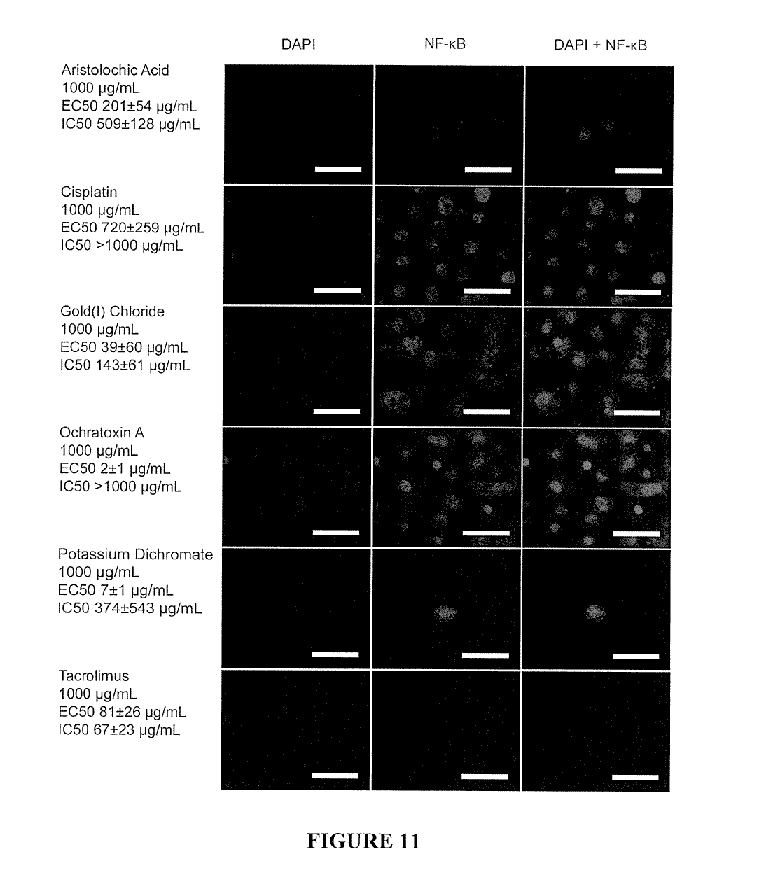

HPTC1 were imaged by HCS after treatment with the indicated compounds, as seen in FIG. 11. The same compounds were used as with respect to FIG. 10. The images in FIG. 11 were captured after treatment with the maximal compound concentrations (1000 μg/ml). Substantial nuclear translocation of NF-κB was observed. On some images only few cells are visible due to cell death. Scale bars: 50 μm.

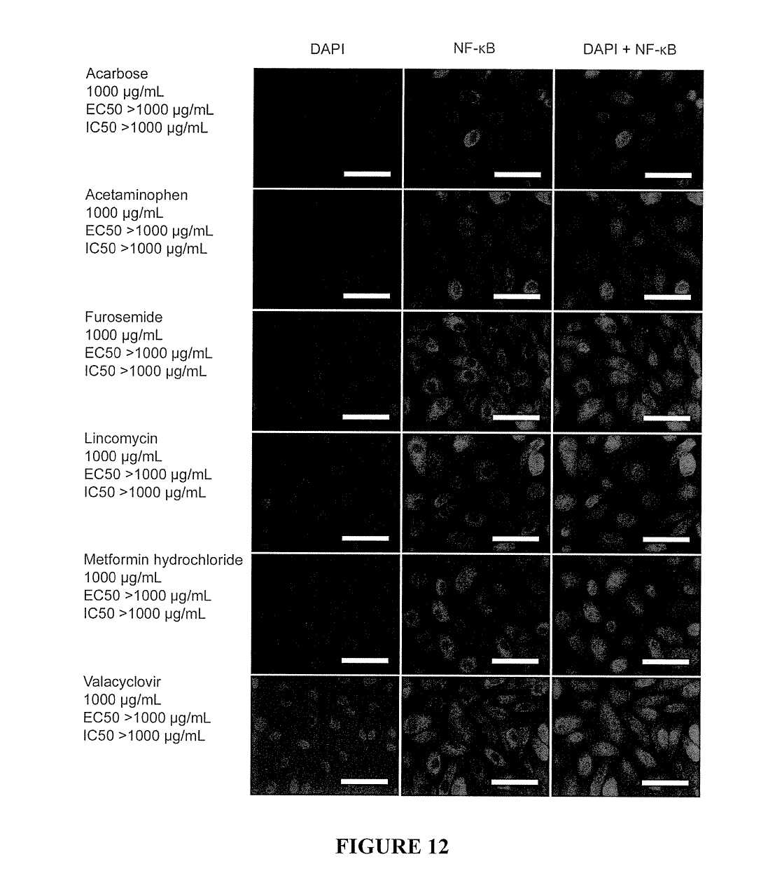

As seen in FIG. 12, HPTC1 were imaged by HCS after treatment with the indicated compounds. The images were captured after treatment with the maximal compound concentrations (1000 μg/mL). All compounds included in this figure are not directly toxic for proximal tubular cells in humans. Substantial nuclear translocation of NF-κB or cell death was not observed. All EC50 and IC50 values were >1000 μg/mL. Scale bars: 50 μm.

Discussion

Although Examples 1 and 2 described above relate to primary and immortalized human renal cells, NF-κB translocation can also be used to screen for toxic effects on other organs by using respective organ-specific cell types. NF-κB is ubiquitously expressed in all human and animal cell types and NF-κB activation occurs in many different organ systems in association with injury, disease and inflammation [7].

Thus, NF-κB translocation could also be used to assess the toxicity and pro-inflammatory capacity of compounds with respect to other organ systems in humans or animals. This could be done by using respective organ-specific human or animal cell types. Preferably, primary cells or similar cell types derived from adult, embryonic or induced pluripotent stem cells should be used.

Thus, organ-specific cell types would be seeded onto substrates suitable for HCS (e.g. multi-well plates). On such substrates, the organ-specific cell type would be treated with the compounds to be screened for potential toxic and pro-inflammatory effects on this cell type. Induction of NF-κB nuclear translocation by the compounds would be determined by HCS and subsequent image analysis, and activation of NF-κB would be classified as positive result. The assays would be performed with primary human or animal organ-specific cell types (e.g. primary renal cell types such as HPTC, primary rat hepatocytes or primary human skin keratinocytes). Alternatively, organ-specific cell types derived from stem cells could be used. Appropriate stem cell types would include all kinds of human or animal adult stem cells (e.g. bone marrow-derived mesenchymal stem cells or adipose-derived stem cells), or human- or animal-derived induced pluripotent stem cells or embryonic stem cells.

Organ systems and cell types that could be included in such NF-κB-based assays are: liver (hepatocytes and other hepatic cell types), kidney (glomerular, tubular and other renal cell types), the cardiovascular system (cardiomyocytes, endothelial cells), the central nervous system (neurons, astrocytes, glia cells), skin (keratinocytes, skin fibroblasts and cell types specific for accessory structures: glands and hair follicles), lung (airway epithelial cells, alveolar cells), pancreas (beta-cells and other pancreatic cell types), digestive tract (different cell types of the stomach and small intestine), cell types specific for reproductive organs including germ cells and their precursors, sensory organs (eye- and ear-specific cell types), bone marrow and blood cells.

Differences between the assay described herein and previously established NF-κB-based HCS assays are as follows. (1) In the assay as described herein, NF-κB/p65 translocation is used as an endpoint to predict the organ-specific toxicity of a compound and its ability to induce NF-κB signaling. This assay does not address modulation of NF-κB translocation that has been induced by an activator/agonist (e.g. TNF-α or IL-1) prior to treatment with the compound being tested. (2) Activators/agonists of NF-κB, such as TNF-α or IL-1, are not used. (3) The HSC assay has been performed with a human primary organ-specific cell type, but could also be performed with differentiated cells derived from stem cells.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

As used in this specification and the appended claims, the singular forms “a”, “an” and “the” include plural reference unless the context clearly dictates otherwise. As used in this specification and the appended claims, the terms “comprise”, “comprising”, “comprises” and other forms of these terms are intended in the non-limiting inclusive sense, that is, to include particular recited elements or components without excluding any other element or component. As used in this specification and the appended claims, all ranges or lists as given are intended to convey any intermediate value or range or any item or sublist contained therein. Unless defined otherwise all technical and scientific terms used herein have the same meaning as commonly understood to one of ordinary skill in the art to which this invention belongs.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the invention.

### REFERENCES

[1] Ben-Neriah Y, Karin M. Inflammation meets cancer, with NF-kappaB as the matchmaker. Nat Immunol. 2011; 12:715-23.
[2] Oeckinghaus A, Hayden M S, Ghosh S. Crosstalk in NF-kappaB signaling pathways. Nat Immunol. 2011; 12:695-708.
[3] Sen R, Baltimore D. Inducibility of kappa immunoglobulin enhancer-binding protein Nf-kappa B by a posttranslational mechanism. Cell. 1986; 47:921-8.
[4] Sun Z, Andersson R. NF-kappaB activation and inhibition: a review. Shock. 2002; 18:99-106.
[5] Oeckinghaus A, Ghosh S. The NF-kappaB family of transcription factors and its regulation. Cold Spring Harb Perspect Biol. 2009; 1:a000034.
[6] Del Prete A, Allavena P, Santoro G, Fumarulo R, Corsi M M, Mantovani A. Molecular pathways in cancer-related inflammation. Biochem Med (Zagreb). 2011; 21:264-75.
[7] Lawrence T. The nuclear factor NF-kappaB pathway in inflammation. Cold Spring Harb Perspect Biol. 2009; 1:a001651.
[8] Guijarro C, Egido J. Transcription factor-kappa B (NF-kappa B) and renal disease. Kidney Int. 2001; 59:415-24.
[9] Sanz A B, Sanchez-Nino M D, Ramos A M, Moreno J A, Santamaria B, Ruiz-Ortega M, et al. NF-kappaB in renal inflammation. J Am Soc Nephrol. 2010; 21:1254-62.
[10] Kunsch C, Lang R K, Rosen C A, Shannon M F. Synergistic transcriptional activation of the IL-8 gene by NF-kappa B p65 (RelA) and NF-IL-6. J Immunol. 1994; 153:153-64.
[11] Kunsch C, Rosen C A. NF-kappa B subunit-specific regulation of the interleukin-8 promoter. Mol Cell Biol. 1993; 13:6137-46.
[12] Matsusaka T, Fujikawa K, Nishio Y, Mukaida N, Matsushima K, Kishimoto T, et al. Transcription factors NF-IL6 and NF-kappa B synergistically activate transcription of the inflammatory cytokines, interleukin 6 and interleukin 8. Proc Natl Acad Sci USA. 1993; 90:10193-7.
[13] Trask O J. Nuclear Factor Kappa B (NF-kappaB) Translocation Assay Development and Validation for High Content Screening. 2004.
[14] Zock J M. Applications of high content screening in life science research. Comb Chem High Throughput Screen. 2009; 12:870-76.
[15] Ding G J, Fischer P A, Boltz R C, Schmidt J A, Colaianne J J, Gough A, et al. Characterization and quantitation of NF-kappaB nuclear translocation induced by interleukin-1 and tumor necrosis factor-alpha. Development and use of a high capacity fluorescence cytometric system. J Biol Chem. 1998; 273:28897-905.
[16] Lin Z, Will Y. Evaluation of drugs with specific organ toxicities in organ-specific cell lines. Toxicol Sci. 2012; 126:114-27.
[17] Wu Y, Connors D, Barber L, Jayachandra S, Hanumegowda U M, Adams S P. Multiplexed assay panel of cytotoxicity in HK-2 cells for detection of renal proximal tubule injury potential of compounds. Toxicol In Vitro. 2009; 23:1170-8.
[18] Li Y, Oo Z Y, Chang S Y, Huang P, Eng K G, Zeng J L, et al. An in vitro method for the prediction of renal proximal tubular toxicity in humans. Toxicol Res. 2013; 2:352-62.
[19] Kodiha M, Brown C, Stochaj U. Analysis of Signaling Events by Combining High-Throughput Screening Technology with Computer-Based Image Analysis. Sci Signal. 2008; 37:12-29.
[20] Li Y, Kandasamy K, Chuah J K, Lam Y N, Toh W S, Oo Z Y, Zink D. Identification of nephrotoxic compounds with embryonic stem-cell-derived human renal proximal tubular-like cells. Mol Pharm. 2014; 11:1982-90.

1. An in vitro method of screening for toxicity of a compound, the method comprising:
contacting a test compound with a test population of cells in which nuclear factor (NF)-κB has not been activated prior to said contacting;
determining nuclear localization levels of NF-κB in the test population subsequent to said contacting; and
comparing the nuclear localization levels of NF-κB in the test population with nuclear localization levels of NF-κB in a control population of cells that has not been contacted with the test compound;
wherein an increase in nuclear localization levels of NF-κB in the test population relative to the control population is indicative that the test compound injures the cells and/or induces a pro-inflammatory response.

2. The method of claim 1, wherein the NF-κB is the p65 subunit of NF-κB.

3. The method of claim 2, wherein the p65 subunit of NF-κB comprises the sequence set forth in any one of SEQ ID NOs: 1 to 4.

4. The method of claim 2, wherein the p65 subunit of NF-κB comprises the sequence set forth in SEQ ID NO: 5 or 6.

5. The method of claim 1, wherein the cells of the test population and control population are human cells or are non-human animal cells.

6. (canceled)

7. The method of claim 1, wherein the cells of the test population and control population comprise stem cells.

8. The method of claim 7, wherein the stem cells comprise embryonic stem cells, mesenchymal stem cells, hematopoietic stem cells, induced pluripotent stem cells, tissue-specific stem cells, or organ-specific stem cells.

9. The method of claim 1, wherein the cells of the test population and control population comprise somatic cells or cells derived from stem cells.

10. The method of claim 9, wherein the cells comprise tumour cells or comprise primary cells.

11. (canceled)

12. The method of claim 9, wherein the cells comprise liver cells, kidney cells, cardiovascular cells, central nervous system cells, skin cells, lung cells, pancreatic cells, digestive tract cells, eye cells, ear cells, bone marrow cells or blood cells.

13. The method of claim 9, wherein the cells comprise renal proximal tubular cells.

14. The method of claim 13, wherein the renal proximal tubular cells are human primary renal proximal tubular cells.

15. The method of claim 9, wherein the cells comprise cells from an established cell line.

16. The method of claim 15, wherein the cells are HK-2 cells, or LLC-PK1 cells.

17. The method of claim 9, wherein the cells comprise cells derived from stem cells which are differentiated from embryonic stem cells, mesenchymal stem cells, induced pluripotent stem cells or organ/tissue-specific stem cells.

18. The method of claim 17, wherein the cells are at least partially differentiated to resemble liver cells, kidney cells, cardiovascular cells, central nervous system cells, skin cells, lung cells, pancreatic cells, digestive tract cells, germ cells or their precursors, eye cells, ear cells, bone marrow cells or blood cells.

19. The method of claim 18, wherein the cells are renal proximal tubular-like cells.

20. The method of claim 1, wherein said contacting is performed over a period of time of from about 1 hour to about 16 hours, from about 12 hours to about 16 hours, or from about 30 to about 36 hours, or for about 3 days.

21. The method of claim 1, further comprising repeating said contacting one or more times, at regular intervals over a total time period of up to 4 weeks, prior to said determining, including wherein said contacting is performed for the same time period for each occurrence of said contacting.

22. The method of claim 1, further comprising repeating said contacting followed by said determining one or more times, at regular intervals over a total time period of up to 4 weeks, Including wherein said contacting is performed for the same time period for each occurrence of said contacting.
